(19) United States
(12) Patent Application Publication
(10) Pub. No.: US 2026/0263385 A1
(43) Pub. Date: Sep. 10, 2026

(54) LUTEIN AND 1,3-DIOLEOYL-2-PALMITOYL GLYCEROL FOR USE IN THE PREVENTION OR TREATMENT OF A DISORDER OF THE GASTROINTESTINAL TRACT

(71) Applicant: N.V. Nutricia, Zoetermeer (NL)

(72) Inventors: Ingrid Brunhilde Renes, Utrecht (NL); Jan Knol, Utrecht (NL); Noortje IJssennagger, Utrecht (NL); Xianfeng Zhao, Shanghai (CN)

(73) Assignee: N.V. Nutricia, Zoetermeer (NL)

(21) Appl. No.: 19/661,775

(22) Filed: Apr. 29, 2026

Related U.S. Application Data
(63) Continuation of application No. PCT/EP2024/080953, filed on Nov. 1, 2024.

(30) Foreign Application Priority Data
Nov. 2, 2023 (WO) PCT/CN2023/129298

(51) Int. Cl. A61K 31/047; A23L 33/00; A61K 31/231; A61K 31/702; A61K 47/26

(52) U.S. Cl. CPC A61K 31/047; A23L 33/40; A61K 31/231; A61K 31/702; A61K 47/26

(57) ABSTRACT

The present invention relates to the combination of lutein and 1,3-dioleoyl-2-palmitoylglycerol.

### FIELD OF THE INVENTION

The invention is in the field of nutrition and more particularly relates to a composition for use in prevention or treatment of a disorder of the gastrointestinal tract in a human subject, in particular disorders of the gastrointestinal tract associated with reduced lactase expression.

### BACKGROUND OF THE INVENTION

Gastrointestinal disorders are common within humans, especially within the Western world where high-fat diets are prevalent. It is estimated that between 60 to 70 million Americans suffer from gastrointestinal problems.

Gastrointestinal problems are often associated with lactose intolerance which is prevalent worldwide in 57 to 65% of all humans. Lactose intolerance is caused by a reduction or loss of the activity of the intestinal enzyme lactase-phlorizin hydrolase (more commonly known as lactase) which is responsible for the digestion of lactose. Said reduction or loss of lactase activity results in increased osmotic load in the small intestine and fermentation of lactose by bacterial flora, which may lead to increased production of short-chain fatty acids and gas. This may be followed by the onset of abdominal pain, diarrhoea, and flatulence.

A frequent cause of lactose malabsorption is lactase non-persistence, a condition in which lactase expression decreases during infancy. In contrast, congenital lactase deficiency (a rare condition that presents with severe symptoms in new-borns) is due to a complete lack of lactase. In addition, lactose malabsorption may be secondary to acquired conditions including small bowel bacterial overgrowth, infectious enteritis (i.e. giardiasis), or mucosal damage due to coeliac disease, inflammatory bowel disease, drugs, gastrointestinal surgery, short bowel syndrome, or radiation enteritis, conditions that lead to either reduction in the of absorptive capacity of sugars or downregulation of lactase expression in the small intestine.

Furthermore, premature infants do not have a mature level of intestinal lactase activity. Early studies demonstrate that even though intestinal lactase activity develops early in gestation, the largest increase in lactase occurs during the third trimester (Antonowicz et al., Development and distribution of lysosomal enzymes and disaccharidases in human fetal intestine, Gastroenterology, 1974, 67, 51-58). Also, digestion of lactose does not appear to be complete in preterm infants (Shulman et al., Absorption of lactose, glucose polymers, or combination in premature infants, J Pediatr, 1995, 127 (4), 626-631).

There is accordingly a need for compositions that prevent or treat disorders of the gastrointestinal tract associated with lactose malabsorption and/or reduced lactase expression.

### SUMMARY OF THE INVENTION

The present inventors have surprisingly found that a combination of lutein and 1,3-dioleoyl-2-palmitoyl glycerol (OPO) increases the expression of lactase in the gastrointestinal tract. In addition, the inventors have also found that the presence of fermentation products of non-digestible oligosaccharides, i.e. short-chain fatty acids (SCFA's), synergistically increase lactase expression in the gastrointestinal tract, when used in combination with lutein and 1,3-dioleoyl-2-palmitoyl glycerol.

Without being bound to theory, the inventors hypothesise that increasing lactase expression in the epithelium of subjects suffering from disorders of the gastrointestinal tract, caused by compromised lactase expression, may result in lactose being tolerated more in the subject's body and gastrointestinal symptoms may be less.

The invention thus pertains to a combination of lutein and 1,3-dioleoyl-2-palmitoyl glycerol (OPO) for use in the prevention or treatment of a disorder of the gastrointestinal tract in a subject, wherein the disorder of the gastrointestinal tract is caused by compromised lactase expression in the gastro-intestinal tract. Preferably the combination is administered orally, more preferably by oral administration of a nutritional composition comprising the combination.

### FIGURES

FIG. 1 shows the effect of SCFAs; lutein; OPO; lutein+OPO, or lutein+OPO+SCFA on lactase mRNA expression in Caco-2 cells compared to a negative control.

### LIST OF PREFFERED EMBODIMENTS

1. An oral composition comprising lutein and 1,3-dioleoyl-2-palmitoyl glycerol (OPO) for use in the prevention or treatment of a disorder of the gastrointestinal tract in a human subject, wherein the disorder of the gastrointestinal tract is caused by compromised lactase expression in the gastro-intestinal tract.
2. The composition for use according to embodiment 1, wherein prevention or treatment of the disorder of the gastrointestinal tract is by increasing the expression of lactase in the gastro-intestinal tract.
3. The composition for use according to embodiment 1 or 2, wherein the composition comprises non-digestible oligosaccharides.
4. The composition for use according to embodiment 3, wherein the non-digestible oligosaccharides are selected from galacto-oligosaccharides (GOS), fructo-oligosaccharides (FOS), human milk oligosaccharides (HMO) or combinations thereof.
5. The composition for use according to any one of the preceding embodiments, wherein the compromised lactase expression is due to lactase non-persistence, being born preterm, coeliac disease, inflammatory bowel disease, short bowel syndrome, intestinal infection, exposure of the subject to chemotherapy, or combinations thereof.
6. The composition for use according to embodiment 5, wherein the compromised lactase expression is due to lactase non-persistence, being born preterm, or combinations thereof.
7. The composition for use according to any one of the preceding embodiments, wherein symptoms of the disorder of the gastrointestinal tract are selected from diarrhea, constipation, flatulence, bloating, abdominal pain or combinations thereof.
8. The composition for use according to any one of the preceding embodiments, wherein the oral composition is a nutritional composition.
9. The composition for use according to any one of the preceding embodiments, wherein the oral composition is an infant formula, follow-on formula or young child formula.
10. The composition for use according to embodiment 9, wherein the oral composition is an infant formula for preterm and/or small-for-gestational age infants.
11. The composition for use according to any one of the preceding embodiments, wherein the composition comprises ≥5 μg lutein per 100 g dry weight of the composition.
12. The composition for use according to any one of the preceding embodiments, wherein the composition comprises 0.5 to 15 g OPO per 100 g dry weight of the composition.
13. The composition for use according to any one of the preceding embodiments, wherein the composition comprises 0.05-25 g non-digestible oligosaccharides per 100 g dry weight of the composition.
14. The composition for use according to any one of the preceding embodiments, wherein the composition comprises 0-75 g lactose per 100 g dry weight of the composition.
15. The composition for use according to any one of the preceding embodiments, wherein the composition comprises digestible carbohydrates, lipids, proteins and an energy content of at least 400 kcal, wherein the composition further comprises, per 100 g based on dry weight of the composition:
a. 5-1000 μg lutein;
b. 0.25-25 g non-digestible oligosaccharides comprising fructo-oligosaccharides and galacto-oligosaccharides; and
c. 0.5-15 g 1,3-dioleoyl-2-palmitoyl glycerol.

### DETAILED DESCRIPTION OF THE INVENTION

A first aspect of the invention pertains to a combination of lutein and 1,3-dioleoyl-2-palmitoyl glycerol (OPO) for use in the prevention or treatment of a disorder of the gastrointestinal tract in a subject, wherein the disorder of the gastrointestinal tract is caused by compromised lactase expression in the gastro-intestinal tract. In a preferred embodiment, the combination is comprised in an oral composition, more preferably a nutritional composition.

In some jurisdictions, the present invention may be defined as a method for the prevention or treatment of a disorder of the gastrointestinal tract in a subject, wherein the disorder of the gastrointestinal tract is caused by compromised lactase expression in the gastro-intestinal tract, the method comprising orally administering to said subject a combination of lutein and 1,3-dioleoyl-2-palmitoyl glycerol (OPO), preferably comprised in an oral composition.

In some jurisdictions, the present invention may be defined as the use of lutein and 1,3-dioleoyl-2-palmitoyl glycerol (OPO) in the manufacture of an oral composition, preferably a nutritional composition, for the prevention or treatment of a disorder of the gastrointestinal tract in a subject, wherein the disorder of the gastrointestinal tract is caused by compromised lactase expression in the gastro-intestinal tract.

In some jurisdictions administering a nutritional composition to a human subject is considered non-therapeutic. In those instances the invention may be worded as defined above by way of a method comprising administering an oral nutritional composition. For clarity, the method can also be defined as a non-therapeutic method. By definition, the words “non-therapeutic” exclude any therapeutic effect.

Preferably, prevention or treatment of the disorder of the gastrointestinal tract is by increasing the expression of lactase in the gastro-intestinal tract, more preferably by increasing the expression of lactase in the small intestine.

The combination of 1,3-dioleoyl-2-palmitoyl glycerol and lutein are in a therapeutically efficient amount.

### Treatment Group

In a preferred embodiment, the human subject is an adult, preferably an adult of at least 50 years old, more preferably of at least 65 years old.

In a preferred embodiment, the human subject is selected from patients which have undergone abdominal surgery, patients that experience postoperative dysfunction of the gut and/or malnourished patients.

In another preferred embodiment, the human subject is an infant or young child, preferably between 0-60 months of age, more preferably from 0 to 36 months of age. More preferably, the human subject is an infant. The term “infant”, as used herein refers to a human subject with an age between 0 and 24 months, preferably between 0 and 12 months and even more preferably between 0 and 6 months.

The infant preferably is a preterm and/or small-for-gestational age (SGA) infant. A preterm infant relates to an infant born before the standard period of pregnancy is completed, i.e. before or on 37 weeks from the beginning of the last menstrual period of the mother. SGA infants refers to infants whose birth weight lies below the 10th percentile for their gestational age. They have usually been the subject of intrauterine growth restriction (IUGR). Preterm and/or SGA infants include low birth weight infants (LBW infants), very low birth weight infants (VLBW infants), and extremely low birth weight infants (ELBW infants). LBW infants are defined as infants with a weight less than 2500 g. VLBW infants as infants with a weight which is less than 1500 g, and ELBW infants as infants with a weight less than 1000 g.

### Gastrointestinal Tract Disorder

The disorder of the gastrointestinal tract is caused by compromised lactase expression in the gastrointestinal tract. Preferably, the compromised lactase expression is due to lactase non-persistence, being born preterm, coeliac disease, inflammatory bowel disease, short bowel syndrome, intestinal infection, exposure of the subject to chemotherapy, or combinations thereof. More preferably, the compromised lactase expression is due to lactase non-persistence, being born preterm, or combinations thereof. Most preferably, the compromised lactase expression is due to lactase non-persistence or due to being born preterm. Worded differently, preferably the combination (and combination comprised within an oral composition) of lutein and OPO, preferably further combined with non-digestible oligosaccharides is for use in treating lactase non-persistence.

Preferably, the symptoms of the disorder of the gastrointestinal tract are selected from diarrhea, constipation, flatulence, bloating, abdominal pain, or combinations thereof. Worded differently, preferably the combination (and combination comprised within an oral composition) of lutein and OPO, preferably further combined with non-digestible oligosaccharides is for use in treating diarrhea, constipation, flatulence, bloating, abdominal pain, or combinations thereof.

In a further preferred embodiment, the combination (and combination comprised within an oral composition) of lutein and OPO, preferably further combined with non-digestible oligosaccharides is for use in treating diarrhea, constipation, flatulence, bloating, abdominal pain, or combinations thereof in preterm infants and/or small-for-gestational age infants.

### Lutein

Preferably, lutein is in the form of free xanthophylls, xanthophyll esters or other chemical forms of lutein. Lutein is found in high quantities in green leafy vegetables such as spinach, kale, and yellow carrots. Lutein may be obtained or isolated by any method recognized by those skilled in the art. For example, lutein may be obtained by extraction from marigolds or other xanthophylls-rich sources, chemical synthesis, fermentation or other biotechnology-derived and enriched xanthophyll sources. A suitable form of lutein is available commercially as e.g. Floraglo® Lutein. The herein defined amounts of lutein refer to free lutein (i.e., equivalent to 100% pure lutein).

The combination according to the invention comprises lutein. In a preferred embodiment, the combination is comprised in an oral composition and preferably lutein is present in an amount ≥5 μg per 100 g dry weight of the composition, preferably 10-1000 μg lutein per 100 g, more preferably 15-750 μg lutein per 100 g, even more preferably 30-500 μg lutein per 100 g dry weight of the composition.

1,3-dioleoyl-2-palmitoyl glycerol (OPO)

The combination according to the invention comprises 1,3-dioleoyl-2-palmitoyl glycerol (OPO). Preferably, the combination is comprised in an oral composition and preferably OPO is present in an amount of 0.5-15 g per 100 g dry weight of the composition, preferably 1-12 g OPO per 100 g, more preferably 2-10 g OPO per 100 g dry weight of the composition.

Suitable commercially available sources of OPO are e.g. from Loders Croklaan under the name Betapol™ or InFat™ from Enzymotec. Alternatively OPO may be prepared in a manner as for instance described in EP 0698078 and/or EP 0758846.

### Non-Digestible Oligosaccharides

Preferably, the combination further comprises non-digestible oligosaccharides. In a preferred embodiment, the combination is comprised in an oral composition and preferably the composition comprises 0.05-25 g of non-digestible oligosaccharides per 100 g dry weight of the composition, even more preferably 0.15-15 g of non-digestible oligosaccharides per 100 g dry weight of the composition.

As used herein, the term “non-digestible oligosaccharides” refers to oligosaccharides which are not digested in the intestine by the action of digestive enzymes present in the human upper digestive tract, e.g. small intestine and stomach, but reach the distal portions of the intestines, such as the colon, intact where they are fermented by the human intestinal microbiota.

Microbial metabolites of non-digestible oligosaccharides include short chain fatty acids (SCFA), for example acetate, propionate, butyrate, lactate, amongst others. The inventors have surprisingly found that the presence of the fermentation products of non-digestible oligosaccharides, i.e. SCFA's, significantly increased lactase expression when used in combination with lutein and 1,3-dioleoyl-2-palmitoyl glycerol.

Preferably the non-digestible oligosaccharides are selected from galacto-oligosaccharides (GOS), fructo-oligosaccharides (FOS), human milk oligosaccharides (HMO), or combinations thereof.

In an embodiment, the non-digestible oligosaccharides are preferably selected from FOS, GOS, or combinations thereof. More preferably, the combination and/or the oral composition comprises FOS and GOS at a weight ratio between (20 to 2): 1, more preferably (16 to 4): 1, even more preferably (12 to 6): 1. Most preferably the weight ratio is about 9:1.

Preferably, the combination comprised within an oral composition comprises FOS, GOS or combinations thereof in an amount of 0.25-25 g per 100 g dry weight of the composition, more preferably 0.5-10 g per 100 g, even more preferably 1.5-7.5 g per 100 g dry weight of the composition.

The GOS preferably are beta-galacto-oligosaccharides. Beta-galacto-oligosaccharides may also be referred to as trans-galacto-oligosaccharides (TOS). GOS is for example sold under the trademark Vivinal™ (Borculo Domo Ingredients, Netherlands) or Bi2Munno (Classado). Preferably the GOS comprises at least 80% beta-1,4 and beta-1,6 linkages based on total linkages, more preferably at least 90%. The GOS preferably has a DP of 2-60, more preferably a DP of 3-40, even more preferably a DP of 4-20 and most preferably a DP of 5-10.

FOS preferably comprises a chain of beta-linked fructose units with a DP of 2 to 250, more preferably 5 to 100, even more preferably 10 to 60. Preferably the FOS has an average DP above 20. FOS may preferably include inulin, levan and/or a mixed type of polyfructan. An especially preferred FOS is inulin. A FOS suitable for use in the compositions is also commercially available, e.g. Raftiline®HP (Orafti).

In an embodiment the non-digestible oligosaccharides preferably are human milk oligosaccharides. “Human milk oligosaccharides” (HMO) are present in human milk and are non-digestible oligosaccharides built from one or more of the following monomers: D-glucose, D-galactose, N-acetylglucosamine, L-fucose and sialic acid (N-acetylneuraminic acid).

The combination comprised within an oral composition preferably comprises HMO in an amount of 0.05-12 g per 100 g dry weight of the composition, more preferably 0.075-9 g per 100 g, even more preferably 0.15-6 g per 100 g, and most preferably 0.3-3 g per 100 g dry weight of the composition.

Preferably, the HMO are selected from sialyloligosaccharides, such as 3′-sialyllactose (3′-SL), 6′-sialyllactose (6′-SL), lactosialyl-tetrasaccharide a,b,c (LST), disialyllacto-N-tetraose (DSLNT), sialyl-lacto-N-hexaose (S-LNH), DS-LNH, and fucooligosaccharides, such as (un) sulphated fucoidan oligosaccharide, 2′-fucosyllactose (2′-FL), 3-fucosyllactose (3-FL), difucosyllactose, lacto-N-fucopenatose, (LNFP) I, II, III, IV Lacto-N-neofucopenaose (LNnFP), Lacto-N-difucosyl-hexaose (LNDH), and combinations thereof.

Preferably, the HMO is selected from 2′-fucosyllactose (2′-FL), 3-fucosyllactose (3-FL), 3-sialyllactose (3′-SL), 6-sialyllactose (6′-SL), lacto-N-tetrose (LNT), lacto-N-neotetrose (LNnT), difucosyllactose (DFL) or combinations thereof. More preferably, the HMO is selected from 2′-FL, 3-FL, LNT, 3′-SL and 6′-SL, or combinations thereof. Most preferably HMO is 2′-FL.

In an embodiment, the non-digestible oligosaccharides preferably comprise GOS and FOS in combination with 2′-FL and/or LNT, most preferably GOS, FOS in combination with 2′-FL.

Preferably, the weight ratio of HMO (preferably 2′-FL) to GOS is from 5 to 0.05, more preferably 5 to 0.1, more preferably from 2 to 0.2. Preferably the weight ratio HMO (preferably 2′-FL) to FOS is from 10 to 0.05, more preferably 5 to 0.1, more preferably from 2 to 0.5.

### Combination

The combination according to the invention consists of lutein and 1,3-dioleoyl-2-palmitoyl glycerol (OPO). In a preferred embodiment, the combination further consists of non-digestible oligosaccharides selected from galacto-oligosaccharides (GOS), fructo-oligosaccharides (FOS), human milk oligosaccharides (HMO) or combinations thereof, more preferably the non-digestible oligosaccharides are selected from galacto-oligosaccharides and fructo-oligosaccharides.

In a further preferred embodiment, the combination according to the invention consists of lutein, OPO and non-digestible oligosaccharides, wherein the non-digestible oligosaccharides are human milk oligosaccharides selected from 2′-fucosyllactose (2′-FL), 3-fucosyllactose (3-FL), 3-sialyllactose (3′-SL), 6-sialyllactose (6′-SL), lacto-N-tetrose (LNT), lacto-N-neotetrose (LNnT), difucosyllactose (DFL) or combinations thereof, more preferably the non-digestible oligosaccharides are GOS and FOS combined with 2′-FL and/or LNT, most preferably GOS, FOS in combination with 2′-FL.

In a preferred embodiment, the combination comprises FOS and GOS at a weight ratio between (20 to 2): 1, more preferably (16 to 4): 1, even more preferably (12 to 6): 1. Most preferably the weight ratio is about 9:1. In a further preferred embodiment, the combination comprises HMO (preferably 2′-FL) and GOS at a weight ratio from 5 to 0.05, more preferably 5 to 0.1, more preferably from 2 to 0.2. Preferably the combination comprises HMO (preferably 2′-FL) and FOS at a weight ratio from 10 to 0.05, more preferably 5 to 0.1, more preferably from 2 to 0.5.

The combination is for use as outlined herein above. In particular the combination is for use in the prevention or treatment of a disorder of the gastrointestinal tract in a human subject, wherein the disorder of the gastrointestinal tract is caused by compromised lactase expression in the gastro-intestinal tract, preferably the use is for the treatment of the disorder of the gastrointestinal tract. In a preferred embodiment, the use is by oral administration of the combination, preferably by oral administration of a nutritional composition comprising the combination.

### Composition

In a preferred embodiment of the invention, the combination is comprised within an oral composition. The composition is preferably a nutritional composition, more preferably an enteral nutritional composition. Preferably, the nutritional composition is a nutritionally complete composition. The composition preferably comprises digestible carbohydrates, lipids, proteins and combinations thereof.

Preferably, the composition comprises digestible carbohydrates. Preferred digestible carbohydrates are lactose, glucose, sucrose, fructose, galactose, maltose, starch and maltodextrin. Preferably, the composition comprises at least 40 g digestible carbohydrates per 100 g of composition, more preferably 45-70 g digestible carbohydrates per 100 g, based on dry weight of the composition.

Preferably, the composition comprises 0-75 g lactose per 100 g dry weight of the composition, more preferably 10-65 g lactose, and most preferably 20-55 g lactose per 100 g dry weight of the composition.

Preferably, the composition comprises protein. Preferred protein sources are dairy protein or plant protein. Preferably, the composition comprises 5-20 g protein per 100 g composition, more preferably 8-15 g protein per 100 g, based on dry weight of the composition.

Preferably, the composition comprises lipid. Preferred lipid sources are vegetable lipids, milk lipids, microbial lipids, fish lipids and combinations thereof. Preferably, the composition comprises 15-35 g per 100 g composition, preferably 20-30 g per 100 g, based on dry weight of the composition.

The composition preferably is a powder comprising at most 5 wt. % of water by weight of the powder. The powder is preferably reconstituted with water or other food grade aqueous liquid, to form a ready-to drink composition in liquid form. Preferably 10-15 gram of this powder is added to 90 ml of liquid to arrive at a ready-to-drink liquid of 100 ml.

Preferably, the oral composition is an infant formula, follow-on formula or young child formula. More preferably, the oral composition is an infant formula for preterm and/or small-for-gestational age infants.

In the present invention, infant formula refers to nutritional compositions, artificially made, intended for infants of 0 to about 4 to 6 months of age and are intended as a substitute for human milk. Typically, infant formulae are suitable to be used as sole source of nutrition. Such infant formulae are also known as starter formula. Follow-on formula for infants starting with at 4 to 6 months of life to 12 months of life are intended to be supplementary feedings for infants that start weaning on other foods. Infant formulae and follow-on formulae are subject to strict regulations, for example for the EU regulations no. 609/2013 and no. 2016/127. In the present context, young child formula refers to nutritional compositions, artificially made, intended for infants of 12 months to 36 months, which are intended to be supplementary feedings for infants. In the context of the present invention, young child formula may also be named growing-up milk.

Preferably, the infant formula, follow-on formula or young child formula and has an energy density of 60 kcal to 70 kcal/100 ml, when in a ready-to-drink form.

In one preferred embodiment, the infant formula, follow-on formula or young child formula and preferably comprises:
3 to 7 g lipid/100 kcal, preferably 4 to 6 g lipid/100 kcal, most preferably 4.5 to 5.5 g lipid/100 kcal,
1.7 to 3.5 g protein/100 kcal, more preferably 1.8 to 3.0 g protein/100 kcal, most preferably 1.8 to 2.5 g protein/100 kcal; and
5 to 20 g digestible carbohydrates/100 kcal, more preferably 6 to 16 g digestible carbohydrates/100 kcal, most preferably 10 to 15 g digestible carbohydrate/100 kcal.

In a preferred embodiment the composition comprises digestible carbohydrates, lipids, proteins and an energy content of at least 400 kcal per 100 g based on dry weight of the composition, wherein the composition further comprises, per 100 g based on dry weight of the composition:
a. 5-1000 μg lutein; and
b. 0.5-15 g 1,3-dioleoyl-2-palmitoyl glycerol.

In a more preferred embodiment, the composition comprises digestible carbohydrates, lipids, proteins and an energy content of at least 400 kcal per 100 g based on dry weight of the composition, wherein the composition further comprises, per 100 g based on dry weight of the composition:
a. 30-500 μg lutein; and
b. 2-10 g 1,3-dioleoyl-2-palmitoyl glycerol.

In an even more preferred embodiment, the composition comprises digestible carbohydrates, lipids, proteins and an energy content of at least 400 kcal per 100 g based on dry weight of the composition, wherein the composition further comprises, per 100 g based on dry weight of the composition:
a. 5-1000 μg lutein;
b. 0.25-25 g non-digestible oligosaccharides comprising fructo-oligosaccharides and galacto-oligosaccharides; and
c. 0.5-15 g 1,3-dioleoyl-2-palmitoyl glycerol.

In a further preferred embodiment, the composition comprises digestible carbohydrates, lipids, proteins and an energy content of at least 400 kcal per 100 g based on dry weight of the composition, wherein the composition further comprises, per 100 g based on dry weight of the composition:

a. 30-500 μg lutein;
b. 0.5-10 g non-digestible oligosaccharides comprising fructo-oligosaccharides and galacto-oligosaccharides; and
c. 2-10 g 1,3-dioleoyl-2-palmitoyl glycerol.

In a further preferred embodiment, the composition comprises digestible carbohydrates, lipids, proteins and an energy content of at least 400 kcal per 100 g based on dry weight of the composition, wherein the composition further comprises, per 100 g based on dry weight of the composition:
a. 5-1000 μg lutein;
b. 0.25-25 g non-digestible oligosaccharides selected from galacto-oligosaccharides (GOS), fructo-oligosaccharides (FOS), human milk oligosaccharides (HMO) or combinations thereof; and
c. 0.5-15 g 1,3-dioleoyl-2-palmitoyl glycerol.

Most preferably, the composition comprises digestible carbohydrates, lipids, proteins and an energy content of at least 400 kcal per 100 g based on dry weight of the composition, wherein the composition further comprises, per 100 g based on dry weight of the composition:
a. 30-5 00 μg lutein;
b. 0.5-10 g non-digestible oligosaccharides, wherein the non-digestible oligosaccharides comprise galacto-oligosaccharides (GOS), fructo-oligosaccharides (FOS) and human milk oligosaccharides (HMO), preferably wherein the non-digestible oligosaccharides are GOS and FOS combined with 2′-FL and/or LNT, preferably GOS, FOS in combination with 2′-FL
c. 2-10 g 1,3-dioleoyl-2-palmitoyl glycerol.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The present invention is illustrated by the following non-limiting examples.

### Examples

### Materials & Methods

### Ingredient Preparation

Concentrated stocks of each tested ingredient were prepared. Short-chain fatty acids (75% sodium acetate/20% sodium propionate/5% sodium butyrate, B5887/P1880/S5636, Sigma-Aldrich) were resuspended in DMEM medium (31053028, Gibco) at a stock concentration of 4 M and applied at 2 mM final concentration. Micelles were used to deliver lutein into the cells. Lutein (PHR1699, Sigma-Aldrich) was prepared at a concentration of 1 mg/mL in 310 mM sodium taurocholate and 64.5 mM phosphatidylcholine. The final concentration applied to the cells was 0.415 μg/mL Lutein. A 2-palmitoyl glycerol MAG (OPO) (75614, Sigma-Aldrich) concentrated stock solution was prepared at 100 mM in ethanol. The final concentration used in the experiments was 100 M. Final concentrations of ingredients were obtained by dissolving the ingredient stock solutions in serum-free DMEM (Gibco™ 31053028) or PBS solution (PBS, 14190144, Gibco; 0.6% BSA; 1.2 mM CaCl₂); 1 mM MgCl₂).

### Cell Culture and Maintenance

A human Caco-2 cell line (HTB-37 ATCC) was used as a model of intestinal epithelium. Cells (passages 50-70) were maintained in a complete growth medium DMEM (High glucose+Glutamax, phenol red, 31966021, Gibco) supplemented with 10% heat-inactivated fetal bovine serum (10270106, Gibco), 1% penicillin-streptomycin (15140-130, Gibco), 1% non-essential amino acids in MEM (MEM NEAA 100×, 11140-035, Gibco), 1% sodium pyruvate in MEM (100 mM stock, 11360-039, Gibco). The cells were grown at 37° C. and 5% CO₂ and were passaged at 80% confluency.

Incubation of Caco-2 Cells with Ingredients

Caco-2 cells were seeded at 2.5×10⁵ cells/mL, 1 ml per well, on 24-well plates (3526, Corning COSTAR®, Corning Inc.) and grown for 21 days. Treatments were prepared by mixing the ingredients in serum-free DMEM (Gibco™ 31053028). Then, the medium on each well was removed and replaced with the treatment (1 ml per well) for 16 h at 37° C. 5% CO₂. Each treatment was performed in triplicate. A medium control was included as a negative control. After 16 h incubation cells were harvested and RNA was extracted.

### RNA Isolation

Total RNA from Caco-2 cells was isolated using TRIzol reagent (15596026, Ambion Life technologies) followed by RNeasy kit from Qiagen, according to the manufacturer's protocol. The quality of the extracted RNA was assessed with Bioanalyzer, RIN was >8 for each sample (Agilent 2100, SoftMax Pro 7.0 software) and the concentration of each sample was measured using Nanodrop 2000 spectrophotometer.

### RNA Sequencing

Paired-end sequence reads were generated at BaseClear (Leiden, the Netherlands) using the Illumina NovaSeq 6000 system. The sequences generated with the NovaSeq 6000 were performed under accreditation according to the scope of BaseClear B.V. (L457; NEN-EN-ISO/IEC 17025). FASTQ read sequence files were generated using bcl2fastq version 2.20 (Illumina). Initial quality assessment was based on data passing the Illumina Chastity filtering. Subsequently, reads containing PhiX control signal were mapped to the PhiX genome using bowtie2 and aligned reads were removed from the fastq files. In addition, reads containing (partial) adapters were clipped (up to a minimum read length of 50 bp) with fastq-mcf from the ea-utils package version 1.04. The second quality assessment was based on the remaining reads using the FASTQC quality control tool version 0.11.8.

The reads were adapter trimmed and quality-checked using Trim_Galore version 0.6.2 . . . . Next, RNA-seq reads were mapped to the genome reference (GCF_000001405.39_GRCh38.p13_genomic) using STAR aligner version 2.7.1 (Dobin et al., STAR: ultrafast universal RNA-seq aligner, Bioinformatics, 2013, 29 (1), 15-21). Based on alignments, gene counts are estimated by featureCounts version 1.6.3 (Liao et al., featureCounts: an efficient general purpose program for assigning sequence reads to genomic features, Bioinformatics, 2014, 30 (7), 923-930). In addition to the alignments (BAM files), results per sample are provided as both raw and TPM-normalised counts.

### Statistical Analysis

Differential gene expression analysis was performed using DESeq2 version 1.22.2 (Love et al., Moderated estimation of fold change and dispersion for RNA-seq data with DESeq2, Genome Biology, 2014, 15:550). Genes with a q-value (false discovery rate)<0.05 were considered differentially expressed.

### Results

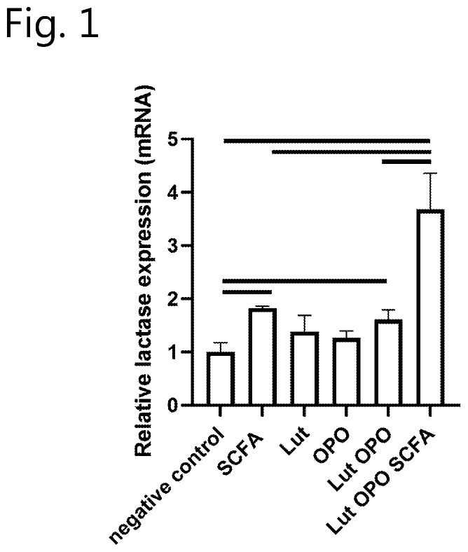

The results are shown in FIG. 1. When comparing to the negative control, the following is observed. The lactase expression in the Caco-2 cells is significantly increased upon stimulation with SCFA. Lactase expression is also significantly increased by the combination of lutein and 1,3-dioleoyl-2-palmitoyl glycerol (OPO). The combination of SCFA, lutein and 1,3-dioleoyl-2-palmitoyl glycerol (OPO) synergistically increased the expression of lactase in the Caco-2 cells.

1. A combination of lutein and 1,3-dioleoyl-2-palmitoyl glycerol (OPO) for use in the prevention or treatment of a disorder of the gastrointestinal tract in a human subject, wherein the disorder of the gastrointestinal tract is caused by compromised lactase expression in the gastro-intestinal tract.

2. The combination for use according to claim 1, wherein the use is for the treatment of the disorder of the gastrointestinal tract.

3. The combination for use according to claim 1 or 2, wherein the use is by oral administration of the combination, preferably by oral administration of a nutritional composition comprising the combination.

4. The combination for use according to any one of the preceding claims, wherein the treatment of the disorder of the gastrointestinal tract is by increasing the expression of lactase in the gastro-intestinal tract.

5. The combination for use according to any one of the preceding claims, wherein the combination further consists ofnon-digestible oligosaccharides selected from galacto-oligosaccharides (GOS), fructo-oligosaccharides (FOS), human milk oligosaccharides (HMO) or combinations thereof.

6. The combination for use according to claim 5, wherein the non-digestible oligosaccharides are selected from GOS and FOS.

7. The combination for use according to claim 5 or 6, wherein the non-digestible oligosaccharides are HMO selected from 2′-fucosyllactose (2′-FL), 3-fucosyllactose (3-FL), 3-sialyllactose (3′-SL), 6-sialyllactose (6′-SL), lacto-N-tetrose (LNT), lacto-N-neotetrose (LNnT), difucosyllactose (DFL) or combinations thereof.

8. The combination for use according to claim 5, wherein the non-digestible oligosaccharides are GOS and FOS combined with 2′-FL and/or LNT, preferably GOSand FOS combined with 2′-FL.

9. The combination for use according to any one of claims 5-8, wherein the combination is comprised in a composition and wherein the composition comprises HMO in an amount of 0.05-12 g per 100 g dry weight of the composition.

10. The combination for use according to claim 5, wherein the weight ratio of HMO to GOS is from 5 to 0.05 and/or wherein the weight ratio of HMO to FOS is from 10 to 0.05.

11. The combination for use according to any one of the preceding claims, wherein the combination is comprised in a composition further comprising 10-65 g lactose per 100 g dry weight of the composition.

12. The combination for use according to any one of the preceding claims, wherein the compromised lactase expression is due to lactase non-persistence, being born preterm, coeliac disease, inflammatory bowel disease, short bowel syndrome, intestinal infection, exposure of the subject to chemotherapy, or combinations thereof.

13. The combination for use according to claim 12, wherein the compromised lactase expression is due to lactase non-persistence, being born preterm, or combinations thereof.

14. The combination for use according to any one of the preceding claims, wherein symptoms of the disorder of the gastrointestinal tract are selected from diarrhea, constipation, flatulence, bloating, abdominal pain or combinations thereof.

15. The combination for use according to claim 3,, wherein the oral administration is by administration of an infant formula, follow-on formula or young child formula.

16. The combination for use according to claim 15, wherein the infant formula is for preterm and/or small-for-gestational age infants.

17. The combination for use according to any one of the preceding claims, wherein the combination is comprised in an oral nutritional composition and wherein the composition comprises digestible carbohydrates, lipids, proteins and an energy content of at least 400 kcal per 100 g based on dry weight of the composition, wherein the composition comprises, per 100 g based on dry weight of the composition:
a. 30-500 μg lutein;
b. 0.5-10 g non-digestible oligosaccharides comprising fructo-oligosaccharides, galacto-oligosaccharides and human milk oligosaccharides; and
c. 2-10 g g 1,3-dioleoyl-2-palmitoyl glycerol.
